(19) United States
(12) Patent Application Publication
(10) Pub. No.: US 2007/0185119 A1
(43) Pub. Date: Aug. 9, 2007

(54) Therapeutic agents II

(75) Inventors: Emma Evertsson, Molndal (SE); Tord Inghardt, Molndal (SE); Jan Lindberg, Molndal (SE); Anna Linusson, Umea (SE)

Correspondence Address: Pepper Hamilton LLP, 500 Grant Street, One Mellon Bank Center, 50th Floor, Pittsburgh, PA, 15219-2502, US

(73) Assignee: ASTRAZENECA AB, Sodertalje (SE)

(21) Appl. No.: 10/596,995

(22) PCT Filed: Jan. 5, 2005
(86) PCT No.: PCT/SE05/00010

§ 371 Date: Nov. 22, 2006

(30) Foreign Application Priority Data
Jan. 7, 2004 (GB) 0400193.9

(51) Int. Cl. A61K 31/517; C07D 403/02; C07D 413/02

(52) U.S. Cl. 514/249; 514/266.2; 544/284

(57) ABSTRACT

Compounds of Formula (I) as well as optical isomers and racemates thereof as well as pharmaceutically acceptable salts, thereof, processes for preparing such compounds, their use in the treatment of obesity, psychiatric disorders, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders and to pharmaceutical compositions containing them.

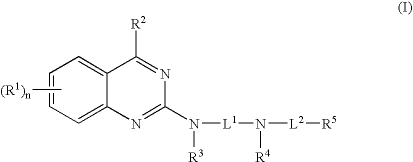

### FIELD OF INVENTION

The present invention relates to certain N-cycloalkyl, aryl or heteroaryl-N′-quinazolin-2-yl cycloalkyldiamines of formula I, to processes for preparing such compounds, to their use in the treatment of obesity, psychiatric and neurological disorders, and to pharmaceutical compositions containing them.

### BACKGROUND OF THE INVENTION

Melanin concentrating hormone (MCH) is a cyclic peptide that was first isolated from fish over 15 years ago. In mammals, MCH gene expression is localized to the ventral aspect of the zona inserta and the lateral hypothalamic area (Breton et al., Molecular and Cellular Neurosciences, vol. 4, 271-284 (1993)). The latter region of the brain is associated with the control of behaviours such as eating and drinking, with arousal and with motor activity (Baker, B., Trends Endocrinol. Metab. 5: 120-126(1994), vol. 5, No. 3, 120-126 (1994)). Although the biological activity in mammals has not been fully defined, recent work has indicated that MCH promotes eating and weight gain (U.S. Pat. No. 5,849,708). Thus, MCH and its agonists have been proposed as treatments for anorexia nervosa and weight loss due to AIDS, renal disease, or chemotherapy. Similarly, antagonists of MCH can be used as a treatment for obesity and other disorders characterized by compulsive eating and excessive body weight. MCH projections are found throughout the brain, including the spinal cord, an area important in processing nociception, indicates that agents acting through MCH1r, such as compounds of formula I, will be useful in treating pain.

Two receptors for MCH (MCH1r (Shimomura et al. Biochem Biophys Res Commun 1999 Aug. 11; 261(3):622-6) & MCH2r (Hilol et al. J Biol Chem. 2001 Jun. 8; 276(23):20125-9)) have been identified in humans, while only one (MCH1r) is present in rodent species (Tan et al. Genomics. 2002 June; 79(6):785-92). In mice lacking MCH1r, there is no increased feeding response to MCH, and a lean phenotype is seen, suggesting that this receptor is responsible for mediating the feeding effect of MCH (Marsh et al. Proc Natl Acad Sci USA. 2002 Mar. 5; 99(5):3240-5). In addition, MCH receptor antagonists have been demonstrated to block the feeding effects of MCH (Takekawa et al. Eur J Pharmacol. 2002 Mar. 8; 438(3):129-35), and to reduce body weight & adiposity in diet-induced obese rats (Borowsky et al. Nat Med. 2002 August; 8(8):825-30). The conservation of distribution and sequence of MCH1r suggest a similar role for this receptor in man and rodent species. Hence, MCH receptor antagonists have been proposed as a treatment for obesity and other disorders characterized by excessive eating and body weight.

U.S. Pat. No. 5,874,438 discloses 2,2′-bridged bis-2,4-diaminoquinazolines are apamine-sensitive potassium channel blockers which are useful in the treatment of dementia, depression, myotonic dystrophy or asthma. N2,N2′-(1,3-cyclohexanediylbis(methylene))bis(N4,N4′-diethyl-2,4-quinazolinediamine is exemplified.

Claim 1 of WO97/20823 was considered to be of very broad scope and vague and therefore unsearchable by the European Patent Office. The search was limited to the Examples. The document discloses quinazolines of formula i 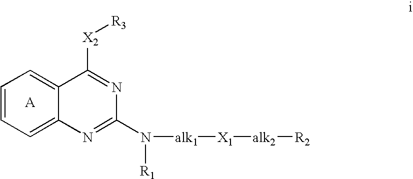
in which X₂ is —O—, S(O)ₙ or a group of the formula —N(R₄), alk₁ is a bond or lower alkylene, X₁ is inter alia C₃-C₈ cycloalkylene or alk₂ is a bond or lower alkylene, R₁ is inter alia H or lower alkyl, R₂ is inter alia substituted amino where the substitution is inter alia by (carbocyclic or heterocyclic) aryl or (carbocyclic or heterocyclic) aryl-loweralkyl, R₃ and R₄ are inter alia H or lower alkyl, and A is a wide range of substituents. The compounds are claimed to be NPY 5 antagonists and therefore useful in the treatment of inter alia obesity and diabetes. Most of the examples are naphthalenesulphonamides, amides or have a 4-anilino substituent in the quinazolyl ring . However, none of the compounds exemplified in this application fall within the scope of the present application. WO 03/028641 discloses that compounds of formula ii
Q-L-Y—R₁ ii
in which inter alia Q is 4-substitutedamino-2-quinazolyl which is unsubstituted in the 5, 6, 7 or 8 positions, L is inter alia 1,4-diaminocyclohexyl wherein there is an optionally an alklylene group between each amine and the cyclohexyl ring, Y is a bond, methylene, carbonyl, or sulphonyl, and R₁ is inter alia heteroaryl are MCH receptor antagonists. Co-pending application WO2004/087680 discloses compounds of formula iii
Q-L-Y—R₁ iii
in which Q is substituted quinazolyl, L is 1,4-diaminocyclohexyl or 1,3-diaminocyclopentyl wherein there is an optionally an alklylene group between each amine and the cycloalkyl ring, Y is C(O)NR, C(S)NR, C(O)O, a bond or CH₂ and R₁ is inter alia phenyl or heterocyclyl are MCH receptor antagonists.

There is an umet need for MCH receptor antagonists that are more potent, more selective, more bioavailable and less toxic than known compounds in this field. The present invention provides additional compounds that are MCH1r antagonists which are useful in treating obesity and related disorders, psychiatric disorders, neurological disorders and pain.

### DESCRIPTION OF THE INVENTION

The present invention relates to a compound of formula I 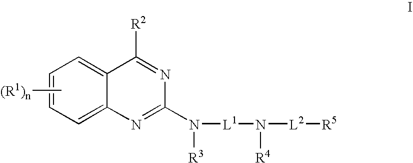
wherein

R¹ represents a) a C₁₋₄alkoxy group optionally substituted by one or more fluoro, b) a C₁₋₄alkyl group optionally substituted by one or more fluoro, c) halo, d) cyano, e) a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O atom f) a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring, or g) a group —OSO₂C₁₋₄alkyl optionally substituted by one or more fluoro;

n represents 0, 1, 2 or 3;

R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;

R³ represents H or a C₁₋₄ alkyl group;

L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O; with the proviso that L¹ does not represent 1,3-cyclopentyl or 1,4-cyclohexyl;

R⁴ represents H or a C₁₋₄ alkyl group optionally substituted by one or more of the following: fluoro or C₁₋₄ alkoxy optionally substituted by one or more fluoro;

L² represents an alkylene chain (CH₂)ₛ in which s represents 1, 2 or 3 wherein the alkylene chain is optionally substituted by one or more of the following: fluoro or C₁₋₄ alkyl;

L² may also represent a 5-6 membered carbocyclic 5-6 membered ring fused to R⁵;

R⁵ represents phenyl or naphthyl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridinyl, 5H-pyrrolo[2,3-b]pyrazinyl, 1H-pyrrolo[3,2-c]pyridinyl, 1H-pyrrolo[2,3-c]pyridinyl, 1H-pyrrolo[2,3-b]pyridinyl, 1H-indazolyl, wherein each R⁵ is optionally substituted by one or more of the following: a) cyano, b) halo, c) a C₁₋₄ alkyl group optionally substituted by one or more fluoro, d) a C₁₋₄ alkoxy group optionally substituted by one or more fluoro, e) a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, f) or by a group (CH₂)_{z}R^{z} in which z and w is 0 or 1 and R^{z} represents phenyl or a heterocyclic group selected from thienyl, pyridyl, thiazolyl, pyrazolyl, wherein each R^{z} is optionally substituted by one or more of the following: cyano, halo, a C₁₋₄ alkyl group optionally substituted by one or more fluoro, or a C₁₋₄alkoxy group optionally substituted by one or more fluoro;

as well as optical isomers and racemates thereof as well as pharmaceutically acceptable salts, thereof.

The invention relates to a compound of the general formula (I) 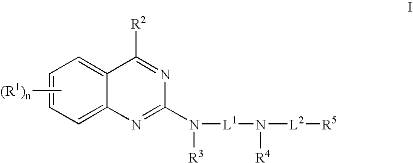
wherein

R¹ represents cyano or a C₁₋₄ alkoxy group optionally substituted by one or more fluoro, a C₁₋₄ alkyl group optionally substituted by one or more fluoro, halo, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O atom, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring,

n represents 0, 1, 2 or 3;

R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;

R³ represents H or a C₁₋₄ alkyl group;

L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or the group —N(R³)-L¹-, or the group L¹-N(R⁴), together represent a saturated heterocyclic ring containing from 2 to 9 carbon atoms and the nitrogen bearing R³ or R⁴, respectively, which may be monocyclic or bicyclic with the proviso that L¹ does not represent 1,3-cyclopentyl or 1,4-cyclohexyl;

R⁴ represents H or a C₁₋₄ alkyl group optionally substituted by one or more of the following: fluoro or C₁₋₄ alkoxy optionally substituted by one or more fluoro;

L² represents an alkylene chain (CH₂)ₛ in which s represents 1, 2 or 3 wherein the alkylene chain is optionally substituted by one or more of the following: fluoro or C₁₋₄ alkyl;

L² may also represent a 5-6 membered carbocyclic 5-6 membered ring fused to R⁵;

R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro as well as optical isomers and racemates thereof as well as pharmaceutically acceptable salts, thereof.

In a particular group of compounds of formula I, L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O or the group —N(R³)-L¹-, or the group L¹-N(R⁴), together represent a saturated heterocyclic ring containing from 2 to 9 carbon atoms and the nitrogen bearing R³ or R⁴, respectively, which may be monocyclic or bicyclic.

In another aspect the invention provides compounds of formula I wherein

R¹ represents cyano or a C₁₋₄ alkoxy group optionally substituted by one or more fluoro, a C₁₋₄ alkyl group optionally substituted by one or more fluoro, halo, cyano, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring,

n represents 0, 1, 2 or 3;

R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;

R³ represents H or a C₁₋₄ alkyl group;

L¹ represents a (CH₂)ₚC₅₋₆ cycloalkyl group in which p is 0 or 1 and provided that there are 3 carbon atoms between the two nitrogens bearing R³ and R⁴, respectively, wherein one of the carbons of the cycloalkyl group may be replaced by O;

R⁴ represents H or a C₁₋₄ alkyl group optionally substituted by one or more of the following: fluoro or C₁₋₄ alkoxy optionally substituted by fluoro;

L² represents an alkylene chain (CH₂)ₛ in which s represents 1, 2 or 3 wherein the alkylene chain is optionally substituted by one or more of the following: fluoro or C₁₋₄ alkyl;

L² may also represent a 5-6 membered carbocyclic 5-6 membered ring fused to R⁵;

R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, as well as optical isomers and racemates thereof as well as pharmaceutically acceptable salts, thereof.

A particular group of compounds of formula I is represented by formula IA 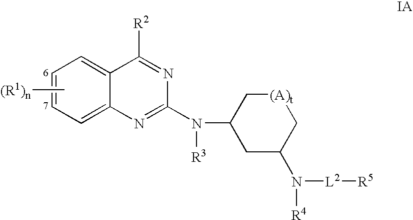
in which

R¹ represents chloro, fluoro, methoxy or a group NR^{a}R^{b} in which R^{a} and R^{b} independently represent a C₁₋₄alkyl group or R^{a} and R^{b} together with the nitrogen atom to which they are attached represent a saturated 3 to 7 membered heterocyclic ring optionally including an O;

n represents 0 or 1, and when n=1 the substituent is attached to either position 6 or 7

R² represents H or cyano or a C₁₋₄alkyl group, a C₁₋₄alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;

R³ represents H;

A represents CH₂ and t is 1;

R⁴ represents H;

L² represents CH₂, C(CH₃)₂ or CF₂; and

R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro as well as optical isomers and racemates thereof as well as pharmaceutically acceptable salts thereof.

Another particular group of compounds of formula I is represented by formula IB 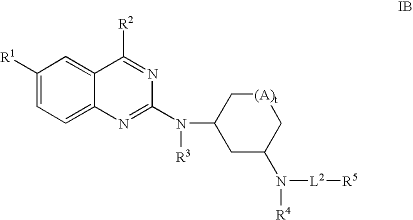
in which

R¹ represents H, cyano, methoxy, isopropoxy, dimethylamino, chloro or fluoro;

R² represents H, cyano, a C₁₋₄alkyl group optionally substituted by one or more fluoro or a C₁₋₄ alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, R³ represents H;

A represents CH₂ and t is 1;

R⁴ represents H;

L² represents CH₂, C(CH₃)₂ or CF₂; and

R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro as well as optical isomers and racemates thereof as well as pharmaceutically acceptable salts thereof.

Another particular group of compounds of formula I is represented by formula IC 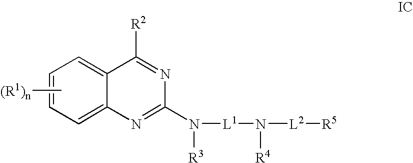
in which R¹ represents cyano or a C₁₋₄ alkoxy group optionally substituted by one or more fluoro, a C₁₋₄ alkyl group optionally substituted by one or more fluoro, halo, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring,

n represents 0, 1, 2 or 3;

R² represents H, cyano, a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;

R³ represents H or a C₁₋₄ alkyl group;

L¹ represents a (CH₂)ₚC₇₋₁₀ cycloalkyl group in which p is 0 or 1 and in which the cycloalkyl group is fused bicyclic or bridged bicyclic provided that the two nitrogens bearing R³ and R⁴, respectively, are not linked to the same carbon atom, and wherein one is of the carbons may be replaced by O;

R⁴ represents H or a C₁₋₄ alkyl group optionally substituted by one or more of the following: fluoro or C₁₋₄ alkoxy, optionally substituted by one or more fluoro;

L² represents an alkylene chain (CH₂)ₛ in which s represents 1, 2 or 3 wherein the alkylene chain is optionally substituted by one or more of the following: fluoro or C₁₋₄ alkyl;

or L² may also represent a 5-6 membered carbocyclic ring fused to R⁵;

R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro as well as optical isomers and racemates thereof as well as pharmaceutically acceptable salts thereof.

Particularly R¹ represents H, cyano, fluoro, methoxy, isopropoxy, chloro or dimethylamino.

Particularly R² represents H, methyl, methoxy, isopropoxy, difluoromethoxy, trifluormethoxy, trifluoromethyl, dimethylamino, 1-pyrrolidinyl or 1-morpholinyl.

Particularly R⁵ represents one or more of the following: 3-thienyl, 1-methylpyrrol-2-yl, 1-methylindol-3-yl, 2,4-dimethoxypyrimidin-5-yl, 2-(phenylsulfonyl)-1,3-thiazol-5-yl, 1-methyl-5-(trifluoromethyl)-1H-pyrazol-3-yl]-2-thienyl, 1-[(2-chloro-1,3-thiazol-5-yl)methyl]-1H-indol-3-yl}, 5-(2-thienyl)thien-2-yl, 5-pyridin-2-yl-2-thienyl, 1,2,3-thiadiazol-4-yl, 4-chloro-1-methyl-1H-pyrazol-3-yl and quinolin-2-yl. Particularly R⁵ represents one or more of the following: 3,4-dichlorophenyl, 6-(trifluoromethyl)pyridin-3-yl, and 2-(trifluoromethoxy)phenyl.

In one particular group of compounds of formula I B , R¹ represents H, fluoro, chloro or dimethylamino; R² represents H, methyl, methoxy, isopropoxy, difluoromethoxy, trifluormethoxy, trifluoromethyl, dimethylamino, 1-pyrrolidinyl or 1-morpholinyl, L² represents CH₂, A is CH₂, t is 1; R³ and R⁴ are each H, and R⁵ represents one of the following: 3-thienyl, 1-methylpyrrol-2-yl, 1-methylindol-3-yl, 2,4-dimethoxypyrimidin-5-yl, 2-(phenylsulfonyl)-1,3-thiazol-5-yl, 1-methyl-5-(trifluoromethyl)-1H-pyrazol-3-yl]-2-thienyl, 1-[(2-chloro-1,3-thiazol-5-yl)methyl]-1H-indol-3-yl}, 5-(2-thienyl)thien-2-yl, 5-pyridin-2-yl-2-thienyl, 1,2,3-thiadiazol-4-yl, 4-chloro-1-methyl-1H-pyrazol-3-yl and quinolin-2-yl.

Particularly in compounds of formula I, p is 0 L¹ is 1,3-cyclohexyl.

More particularly in compounds of formula I, IA, IB and IC the stereochemistry of the cycloalkyl carbon atoms to which the nitrogen atoms are attached is S,S.

Particularly in compounds of formula I, L¹ is selected from:

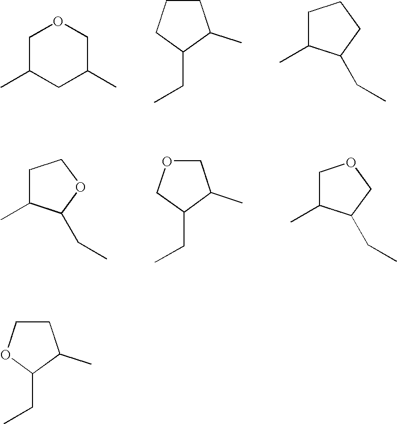

It will be understood that in the above the free bond to the left of the page is attached to the nitrogen bearing R³ and the free bond to the right of the page is attached to the nitrogen bearing R⁴. For the avoidance of doubt when Q represents 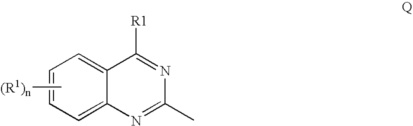
particular compounds of the invention are 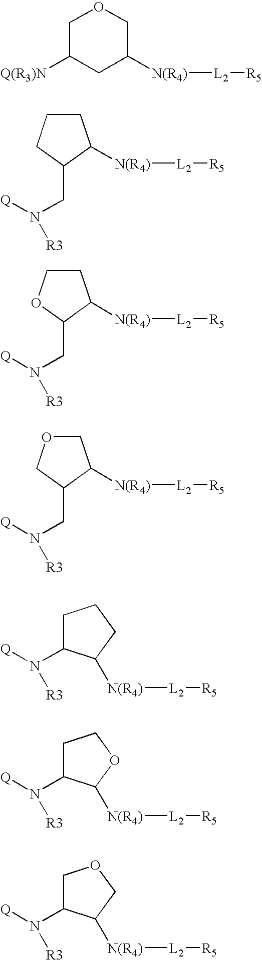
in which Q, R³, R⁴, L² and R⁵ are as previously defined.

In a particular group of compounds of formula I, L¹ represents a (CH₂)ₚC₇₋₁₀ cycloalkyl group in which p is 0 or 1 and in which the cycloalkyl group is fused or bicyclic and optionally may be bridged provided that the two nitrogens bearing R³ and R⁴, respectively, are not linked to the same carbon atom, and wherein one of the carbons may be replaced by O or, alternatively, the group —N(R³)-L¹- or the group L¹-N(R⁴) together represent a saturated bicyclic heterocyclic ring containing from 2 to 9 carbon atoms and the nitrogen bearing R³ or R⁴ respectively.

Alternatively, N(R³)-L¹-N(R⁴) are joined together in a bicylic ring containing 6 to 8 carbon atoms and R¹, R², R³, R⁴, R⁵, L², m and n are as defined above.

Examples where L¹ is bicyclic include

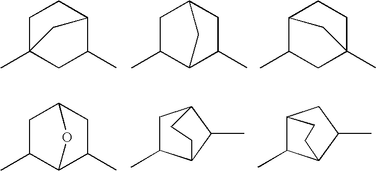

It will be understood that in the above the free bond to the left of the page is attached to the nitrogen bearing R³ (or to the quinoline ring) and the free bond to the right of the page is attached to the nitrogen bearing R⁴ (or to L2 or to R5).

For the avoidance of doubt when Q represents 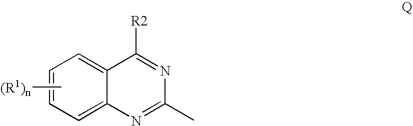
examples of compounds where L¹ is bicyclic include 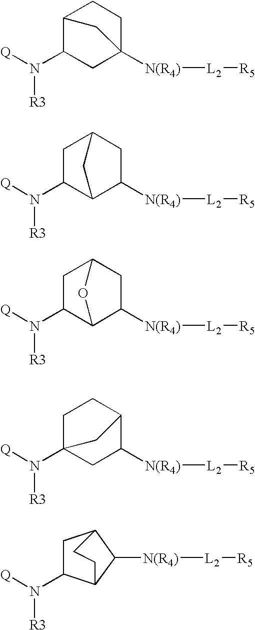
in which Q, R³, R⁴, L² and R⁵ are as previously defined.

The term “pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in is different proportions of the separate enantiomers, where such isomers and enantiomers exist, as well as pharmaceutically acceptable salts thereof. Isomers may be separated using conventional techniques, e.g. chromatography or fractional crystallization. The enantiomers may be isolated by separation of racemate for example by fractional crystallization, resolution or HPLC. The diastereomers may be isolated by separation of isomer mixtures for instance by fractional crystallization, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

Aryl means phenyl or naphthyl in definitions of R⁵ each of which is optionally substituted as described above.

Examples of a C₁₋₄ alkoxy group optionally substituted by one or more fluoro, include trifluoromethoxy, difluoromethoxy, fluoromethoxy and 4,4,4-trifluorobutoxy.

Examples of a C₁₋₄ alkyl group optionally substituted by one or more fluoro include trifluoromethyl, difluoromethyl and fluoromethyl.

Examples of a group OSO₂C₁₋₄alkyl, wherein the alkyl group is optionally substituted with one or more fluorine atoms include methylsulfonyloxy, ethylsulfonyloxy, n-propylsulfonyloxy, n-butylsulfonyloxy, 4,4,4-trifluorobutyl-1-sulfonyloxy and 3,3,3-trifluoropropyl-1-sulfonyloxy.

Examples of a group NR^{a}R^{b} in which R^{a} and R^{b} independently represent H or a C₁₋₄alkyl group include methylamino, ethylamino, propylamino, isopropylamino, butylamino dimethylamino, diethylamino, N-ethyl-N-methylamino and diisopropylamino.

Examples of a group NR^{a}R^{b} in which R^{a} and R^{b} together with the nitrogen atom to which they are attached represent a saturated 3 to 7 membered heterocyclic ring optionally including an O include pyrrolidino, morpholino and piperidino.

Examples of a group CONR^{c}R^{d} in which R^{c} and R^{d} independently represent H or a C₁₋₄alkyl group include N-methylcarbamoyl, N-ethylcarbamoyl, N-propylcarbamoyl, N,N-dimethylcarbamoyl, N-ethyl-N-methylcarbamoyl and N,N-diethylcarbamoyl

Examples of a group CONR^{c}R^{d} in which R^{c} and R^{d} together with the nitrogen atom to which they are attached represent a saturated 3 to 7 membered heterocyclic ring include pyrrolidinocarbonyl and piperidinocarbonyl.

Specific compounds of the invention are
N-(4-methylquinazolin-2-yl)-N′-(3-thienylmethyl)-trans-cyclohexane-1,3-diamine;
N⁴,N⁴-dimethyl-N²-{-3-[(3-thienylmethyl)amino]-trans-cyclohexyl}quinazoline-2,4-diamine;
N²-{-3-[(1-benzothien-3-ylmethyl)amino]-trans-cyclohexyl}-N⁴,N⁴-dimethylquinazoline-2,4-diamine;
N⁴,N⁴-dimethyl-N²-(-3-{[(1-methyl-1H-indol-3-yl)methyl]amino}-trans-cyclohexyl)quinazoline-2,4-diamine;
N⁴,N⁴-dimethyl-N-2-((1S,3S)-3-{[2-(trifluoromethoxy)benzyl]amino}cyclohexyl)-quinazoline-2,4-diamine;
N⁴,N⁴-dimethyl-N-2-[(1S,3S)-3-({[6-(trifluoromethyl)pyridin-3-yl]methyl}amino)-cyclohexyl]quinazoline-2,4-diamine; and
N²-{(1S,3S)-3-[(3,4-dichlorobenzyl)amino]cyclohexyl}-N⁴-N⁴-dimethylquinazoline-2,4-diamine;
   and pharmaceutically acceptable salts thereof.
   Methods of Preparation

The compounds of the invention may be prepared as outlined below according to any of the following methods. However, the invention is not limited to these methods, the compounds may also be prepared as described for structurally related compounds in the prior art.

Compounds of formula I may be prepared by reacting a compound of formula II 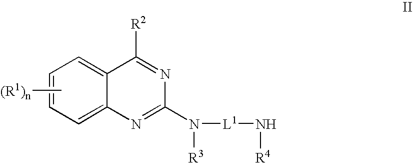
in which R¹, R², R³, R⁴, L¹, and n are as previously defined with an aldehyde or a ketone of formula III
R⁵-L^{2′}=O III
in which R⁵ is as previously defined and L^{2′} represents a group which after reaction of compounds II and III gives L² on reduction, under reductive alkylation conditions. For example, a compound of formula II and a compound of formula III may be reacted together at a temperature in the range of 0° C. to 250° C., preferably in the range of 50° C. to 150° C., optionally in the presence of an inert solvent, for example methanol, dichloromethane or acetic acid in the presence of a reducing agent, for example sodium cyanoborohydride or optionally polymer supported cyanoborohydride.

Compounds of formula II may be prepared by reacting a compound of formula IV 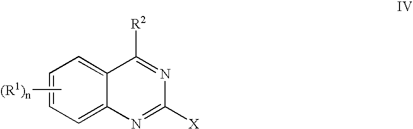
in which R¹, R², and n are as previously defined and X is halo, particularly chloro or bromo, with a compound of formula V 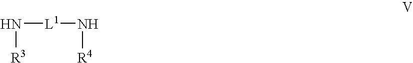
at a temperature in the range of 0° C. to 250° C., preferably in the range of 50° C. to 150° C. in pyridine or optionally in the presence of an inert solvent, for example toluene or dioxane in the presence of a catalytic cross-coupling system for example Pd(OAc)₂ and 2-(di-^{t}butylphosphino)biphenyl or BINAP, and optionally in the presence of a base for example NaO^{t}Bu or Cs₂CO₃.

Certain compounds of formula II and V are novel and are claimed as a further aspect of the present invention as useful intermediates.

Optionally one or both nitrogens in formula V may be protected prior to reaction with a compound of formula IV and then the compound of formula II obtained is deprotected prior to reaction with a compound of formula III. Amine protecting groups are known to those skilled in the art for example the t-Boc, Cbz or phtalimido groups.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

The expression “inert solvent” refers to a solvent which does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free acid, or a pharmaceutically acceptable organic or inorganic base addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

The compounds of the invention may also be combined with other therapeutic agents which are useful in the treatment of disorders associated with obesity, psychiatric disorders, neurological disorders and pain.

Pharmacological Properties

The compounds of formula (I) are useful for the treatment of obesity, psychiatric disorders such as psychotic disorders, anxiety, anxio-depressive disorders, depression, cognitive disorders, memory disorders, schizophrenia, epilepsy, and related conditions, and neurological disorders such as dementia, multiple sclerosis, Raynaud's syndrome, Parkinson's disease, Huntington's chorea and Alzheimer's disease. The compounds are also potentially useful for the treatment of immune, cardiovascular, reproductive and endocrine disorders, and diseases related to the respiratory and gastrointestinal systems. The compounds are also potentially useful as agents for ceasing consumption of tobacco, treating nicotine dependence and/or treating nicotine withdrawal symptoms, reducing the craving for nicotine and as anti-smoking agents. The compounds may also eliminate the increase in weight that normally accompanies the cessation of smoking. The compounds are also potentially useful as agents for treating or preventing diarrhea. The compounds are also potentially useful as agents for reducing the craving/relapse for addictive substances that include, but are not limited to psychomotor-active agents such as nicotine, alcohol, cocaine, amphetamines, opiates, benzodiazepines and barbiturates. The compounds are also potentially useful as agents for treating drug addiction and/or drug abuse.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

Accordingly, it is desirable to provide a compound and method of treatment which will be active in reducing craving for the abused substance, and which does not exacerbate the sympathetic response rate caused by the abused substance and which has favorable pharmacodynamic effects.

The compounds are also potentially useful as agents for treating pain disorders, including but not limited to acute and chronic nociceptive, inflammatory and neuropathic pain and migraine.

In another aspect the present invention provides a compound of formula I as claimed in any previous claim for use as a medicament.

In a further aspect the present invention provides the use of a compound of formula I in the preparation of a medicament for the treatment or prophylaxis of obesity, psychiatric disorders such as psychotic disorders, anxiety, anxio-depressive disorders, depression, bipolar disorder, ADHD, cognitive disorders, memory disorders, schizophrenia, epilepsy, and related conditions, neurological disorders such as dementia, multiple sclerosis, Parkinson's disease, Huntington's chorea and Alzheimer's disease and pain related disorders , including but not limited to acute and chronic nociceptive, inflammatory and neuropathic pain and migraine, comprising administering a pharmacologically effective amount of a compound of formula I to a patient in need thereof.

In a still further aspect the present invention provides a method of treating obesity, psychiatric disorders such as psychotic disorders, anxiety, anxio-depressive disorders, depression, bipolar disorder, ADHD, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I to a patient in need thereof.

The compounds of the present invention are particularly suitable for the treatment of obesity.

In another aspect the present invention provides a method of treating obesity, type II diabetes, Metabolic syndrome and a method of preventing type II diabetes comprising administering a pharmacologically effective amount of a compound of formula I to a patient in need thereof.

Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. For example, a compound of the present invention may be used in combination with a compound that affects thermogenesis, lipolysis, fat absortion, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to microangiopathies.

The compounds of the invention may be used alongside other therapies for the treatment of metabolic syndrome or type 2 diabetes and its associated complications, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt, solvate, solvate of such a salt or a prodrug thereof, may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

According to an additional further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-obesity compound for example orlistat (EP 129,748) and sibutramine (GB 2,184,122 and U.S. Pat. No. 4,929,629);
an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;
a CB1 antagonist or inverse agonist;
another Melanin concentrating hormone (MCH) antagonist;
a PDK inhibitor; or
modulators of nuclear receptors for example LXR, FXR, RXR, and RORalpha;
an SSRI;
a serotonin antagonist;
   or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for for the treatment of type 2 diabetes and its associated complications in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof.

According to a further aspect of the present invention there is provided a kit comprising:
a) a compound of formula I, or a pharmaceutically acceptable salt, solvate, solvate of such a salt or a prodrug thereof, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to a further aspect of the present invention there is provided a kit comprising:
a) a compound of formula I, or a pharmaceutically acceptable salt, solvate, solvate of such a salt or a prodrug thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the the treatment of metabolic syndrome or type 2 diabetes and its associated complications in a warm-blooded animal, such as man.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

### WORKING EXAMPLES

The invention will now be described in more detail with the following examples that are not to be construed as limiting the invention.

### ABBREVIATIONS

aq. aqueous
Ac acetyl
BINAP rac-2,2′-Bis(diphenyl-phosphino)-1,1′-binaphtyl
Bu butyl
DCM dichloromethane
DMF N,N-dimethylformamide
ELS evaporative light scattering
Et ethyl
HEK human embryotic kidney
HPLC high performance liquid chromatography
LC liquid chromatography
MS mass spectroscopy
Pol-BH₃CN (polystyrylmethyl)trimethylammonium cyanoborohydride (loading 4.1-4.3 mmol BH₃CN/g)
Pol-CHO 4-benzyloxybenzaldehyde polystyrene (loading ˜2.66 mmol CHO/g)
TFA trifluoroacetic acid
THF tetrahydrofaran
TLC thin layer chromatography
Tris trishydroxymethylaminomethane
t tert
rt. room temperature
sat. saturated
br broad
bs broad singlet
bt broad triplet
d doublet
dd doublet of doublets
m multiplet
q quartet
s singlet
t triplet
tt triplet of triplets
td triplet of doublets
bd broad doublet

### General Experimental Procedures

Flash column chromatography employed MERCK normal phase silica gel 60 Å (40-63 μm) or a Biotage Horizon Pioneer® HPFC system equipped with FLASH 12+M or FLASH 25+M or 40+M silica cartridges. Mass spectra were recorded on a Waters Micromass ZQ single quadrupole equipped with a pneumatically assisted electrospray interface (LC-MS). Purifications were performed on a Waters Prep LC 2000 with UV-detection, equipped with a Kromasil 10 μm C8 250 mm×20 mm column, or on a semi preparative HPLC, Shimadzu LC-8A, Shimadzu SPD-10A UV-vis.-detector equipped with a Waters Symmetry® 100 mm×19 mm C18 5 μm column.

Automated HPLC purification was done using a Waters Fraction Lynx system equipped with UV, ELS and MS detection and an Ace C8 5μ 10 cm×21.2 id column. The mobile phase was A: 95% CH₃CN and B: 5% CH₃CN+95% 0.1 M NH₄OAc with a gradient from 100% B to 100% A in 10 minutes at 25 mL/min flow rate.

¹H NMR and ¹³C NMR spectra were obtained at 298 K on a Varian Unity Plus 400 mHz, or a Varian Inova 500 MHz or a Varian Unity Plus 600 MHz or a Bruker Avance 300 MHz. Chemical shifts are given in ppm with the solvent residual peak as internal standard: CDCl₃ δ_{H} 7.26, δ_{C} 77.2; MeOH-d₄ δ_{H} 3.31, δ_{C} 49.0; DMSO-d₆ δ_{H} 2.50; δ_{C} 39.5 ppm. Microwave heating was performed using single node heating in a Smith Creator from Personal Chemistry, Uppsala, Sweden.

Analytical chiral HPLC was done using a Chiralcel OJ (250×4.6 mm i.d.) column with EtOH:Et₃N 100:0.1 as mobile phase at flow rate 1 mL/min and with UV detection at 254 or 350 nm.

Names/reference numbers of starting materials (CAS no), either commercially available or prepared by published methods.

2-chloro-4-methylquinazoline, 6141-14-6; cyclohexane-1,3-diamine, 3385-21-5; 3-thiophenecarbaldehyde, 498-62-4; benzo[b]thiophene-3-carbaldehyde, 5381-20-4; 1-methylindole-3-carbaldehyde, 19012-03-4; rac-2,2′-bis(diphenylphosphino)-1,1′-binaphthyl (BINAP), 98327-87-8; 2-trifluoromethoxybenzaldehyde, 94651-33-9; 6-(trifluoromethyl)nicotinaldehyde, 386704-12-7; 3,4-dichlorobenzaldehyde, 6287-38-3.

Preparation of Intermediates

### Dibenzyl trans-cyclohexane-1,3-diylbiscarbamate

D-tartaric acid (15.77 g, 105 mmol) was added to a stirred solution of cyclohexane-1,3-diamine (12 g, 105 mmol, cis/trans ˜2.6:1) in H₂O (80 mL). The resulting mixture was heated to ˜60° C. and MeOH (800 mL) was slowly added. The mixture was allowed to attain rt and left for 3 days. The precipitate was filtered off and the filtrate was concentrated and redissolved in 1M NaOH (40 mL). To the stirred mixture at 0° C. was added benzyl chloroformate (9.56 g, 56 mmol) and 1M NaOH (40 mL). After 5 min, 1,4-dioxane (40 mL) was added and the mixture stirred for an additional 18 h at rt. The mixture was diluted with H₂O and extracted with CH₂Cl₂. The organic layer was dried with MgSO₄, filtered and concentrated. Purification on a Biotage Horizon 40+M SiO₂ column gave 5.61 g (14%) of the title compound as a white solid.

¹H NMR (400 MHz, MeOH-d₄), δ 7.36-7.26 (m, 5H), 5.06 (bs, 2H), 3.77 (b, 2H), 1.73-1.42 (m, 8H). LC-MS [M+H]⁺383.4.

### (+) Dibenzyl-trans-cyclohexane-1,3-diylbiscarbamate

The enantiomers of dibenzyl-trans-cyclohexane-1,3-diylbiscarbamate were separated by preparative chiral chromatography. 7.27 g were dissolved in EtOH (56 mg/mL), repeated 2 mL (112 mg) injections on a Chiralcel OJ (250×20 mm i.d.), eluted with EtOH:Et₃N 100/0.1, 12 mL/min, gave 3.75 g of the title compound, 99.3% ee, [α]²⁰_{D}+2.7 (c 1.26, MeOH) and 2.45 g of (−)dibenzyl-trans-cyclohexane-1,3-diylbiscarbamate, 83% ee.

### (1S,3S)-Cyclohexane-1,3-diamine dihydrochloride

(+)dibenzyl-trans-cyclohexane-1,3-diylbiscarbamate (0.24 mmol, 0.090g) and 10% Pd on activated carbon (0.010 g) in EtOH (5 mL) was stirred under a H₂-atmosphere. After 1 h, the mixture was filtered through Celite and concentrated to give 44 mg of the title compound (100%). The product was recrystallized from MeOH/Et₂O and the absolute configuration was determined by X-ray crystallography.

### EXAMPLES

### Example 1

### N-(4-methylquinazolin-2-yl)-N′-(3-thienylmethyl)-trans-cyclohexane-1,3-diamine

### a) Dibenzyl trans-cyclohexane-1,3-diylbiscarbamate

D-tartaric acid (15.77 g, 105 mmol) was added to a stirred solution of cyclohexane-1,3-diamine (12 g, 105 mmol, cis/trans ˜2.6:1) in H₂O (80 mL). The resulting mixture was heated to ˜60° C. and MeOH (800 mL) was slowly added. The mixture was allowed to attain rt and left for 3 days. The precipitate was filtered off and the filtrate was concentrated and redissolved in 1M NaOH (40 mL). To the stirred mixture at 0° C. was added benzyl chloroformate (9.56 g, 56 mmol) and 1M NaOH (40 mL). After 5 min, 1,4-dioxane (40 mL) was added and the mixture stirred for an additional 18 h at rt. The mixture was diluted with H₂O and extracted with CH₂Cl₂. The organic layer was dried with MgSO₄, filtered and concentrated. Purification on a Biotage Horizon 40+M SiO₂ column gave 5.61 g (14%) of the title compound as a white solid.

¹H NMR (400 MHz, MeOH-d₄), δ 7.36-7.26 (m, 5H), 5.06 (bs, 2H), 3.77 (b, 2H), 1.73-1.42 (m, 8H). LC-MS [M+H]⁺383.4.

### b) Benzyl(3-{benzyloxycarbonyl-[4-methylquinazolin-2-yl]amino}-trans-cyclohexyl)carbamate

A mixture of 2-chloro-4-methylquinazoline (1.70 g, 9.57 mmol), dibenzyl-trans-cyclohexane-1,3-diylbiscarbamate (4.0 g, 10.5 mmol), Cs₂CO₃ (6.96 g, 21.38 mmol), Pd(OAc)₂ (0.213 g, 0.95 mmol), and BINAP (0.592 g, 0.95 mmol) in toluene/THF (23 mL/10 mL) was stirred at 90° C. under an atmosphere of nitrogen until LC/MS indicated that starting material was consumed. The reaction mixture was cooled to room temperature, diluted with MeOH and filtered through celite. The filtrate was evaporated to dryness. The residue was purified on a SiO₂ column eluted with heptane:EtOAc (1:1) to give 0.840 g of the title compound as a mixture of the bis- and mono-Cbz-protected compound.

LC-MS [M+H]⁺525 and 391.

### c) N-(4-methylquinazolin-2-yl)-trans-cyclohexane-1,3-diamine

Benzyl(3-{benzyloxycarbonyl-[4-methylquinazolin-2-yl]amino}-trans-cyclohexyl)carbamate (1.25 g, 2.38 mmol) was dissolved in MeOH (50 mL). Pd—C (10%, containing 57.7% H₂O) (250 mg) was added and mixture was stirred at room temperature under a hydrogen atmosphere until LC-MS indicated that starting material was consumed. The reaction mixture filtered through celite and evaporated to dryness. The residue was dissolved in MeCN and purified by HPLC (Eluent A: H₂O containing 0.1% TFA; Eluent B: MeCN; gradient from 5% to 85% of eluent B) to give 0.306 g (50%) of the title compound.

¹H NMR (300.1 MHz, CDCl₃), δ 7.81-7.84 (d, 1H), 7.54-7.65 (m, 2H), 7.16-7.21 (m, 1H), 5.23 (br d, 1H), 4.45 (br s, 1H), 3.11 (m, 1H), 2.74 (s, 3H), 1.60-2.04 (m, 8H), 1.25-1.33 (m, 1H). ¹³C NMR (CDCl₃), δ 169.4, 158.3, 152.1, 133.7, 126.3, 125.4, 122.2, 119.7, 46.3, 46.2, 40.4, 35.2, 30.9, 21.7, 20.0. LC-MS [M+H]⁺257.

### d) N-(4-methylquinazolin-2-yl)-N′-(3-thienylmethyl)-trans-cyclohexane-1,3-diamine

N-(4-methylquinazolin-2-yl)-trans-cyclohexane-1,3-diamine (51 mg, 0.2 mmol), thiophene-3-carboxaldehyde (22 mg, 0.2 mmol) and sodium triacetoxyborohydride (90 mg, 0.4 mmol) was added to CH₂Cl₂ (5 ml). The mixture was stirred at rt for 48 h. All starting material was then consumed according to LC-MS so the reaction was quenched with saturated NH₄Cl and the mixture washed with water. The organic phase was separated and the solvent evaporated. The residue was was purified on a pre-packed Si-column (Isolute, 5 g) eluted with CH₂Cl₂/MeOH 10:1 to give 20 mg (28%) of the title product.

¹H NMR (400 MHz, MeOH-d₄), δ 7.97 (d, 1H), 7.68 (t, 1H), 7.55 (d, 1H), 7.42-7.36 (m, 2H), 7.26 (t, 1H), 7.13 (d, 1H), 4.43 (m, 1H), 4.17 (d, 2H), 3.30-3.24 (m, 1H), 2.77 (s, 3H), 2.51-2.43 (m, 1H), 2.13-2.06 (m, 1H), 1.88-1.72 (m, 5H), 1.62-1.51 (m, 1H), ¹³C NMR (101 MHz, MeOH-d₄), δ 170.2, 158.2, 151.6, 134.0, 133.3, 127.8, 126.8, 126.0, 125.6, 125.2, 122.6, 119.5, 52.6, 45.8, 42.9, 32.8, 29.6, 28.9, 20.5, 19.4 LC-MS [M+H]⁺353.0.

### Example 2

### N⁴,N⁴-dimethyl-N²-{-3-[(3-thienylmethyl)amino]-trans-cyclohexyl}quinazoline-2,4-diamine

### a) Benzyl(3-{benzyloxycarbonyl-[4-(dimethylamino)quinazolin-2-yl]amino}-trans-cyclohexyl)carbamate

A mixture of (2-chloro-quinazolin-4-yl)-dimethyl-amine (2.73 g, 13.12 mmol, preparation see WO03028641), dibenzyl-trans-cyclohexane-1,3-diylbiscarbamate (5.52 g, 14.43 mmol, preparation see Example 1a), Cs₂CO₃ (9.62 g, 30 mmol), Pd(OAc)₂ (0.295 g, 1.31 mmol), and BINAP (0.817 g, 1.31 mmol) in toluene:THF (25 mL:13 mL) was stirred at 90° C. under nitrogen until LC-MS indicated that starting material was consumed. The reaction mixture was cooled to room temperature, diluted with MeOH (200 mL) and filtered through celite. The filtrate was then evaporated to dryness. The residue was purified on a SiO₂ column eluted with Heptane:EtOAc (1:1) to give 2.61 g, 4.71 mmol (36% yield) of the title compound.

LC-MS [M+H]⁺554.

### b) N²-(3-amino-trans-cyclohexyl)-N⁴,N⁴-dimethyl-quinazoline-2,4-diamine

Benzyl(3-{benzyloxycarbonyl-[4-(dimethylamino)quinazolin-2-yl]amino}-trans-cyclohexyl)carbamate (2.61 g, 4.71 mmol) was dissolved in MeOH (50 mL). Pd—C (10%, containing 57.7% H₂O) (500 mg) was added and the mixture was stirred at room temperature under a hydrogen atmosphere until LC-MS indicated that starting material was consumed. The reaction mixture was filtered through Celite and evaporated to dryness to give 1.12 g (83%) of the title compound.

¹H NMR (300.1 MHz, CDCl₃), δ 7.78-7.80 (d, 1H), 7.43-7.51 (m, 2H), 6.70-7.05 (m, 1H), 4.32 (br, 1H), 3.28 (s, 6H), 3.11 (m, 1H), 1.90 (m, 1H), 1.51-1.71 (m, 7H), 1.23-1.25 (m, 1H). ¹³C NMR (75.5 MHz, CDCl₃), δ 170.7, 164.8, 156.9, 133.0, 126.4, 123.7, 120.7, 111.8, 46.7, 46.4, 42.2, 40.4, 35.2, 31.2, 20.1. LC-MS [M+H]⁺286.

### c) N⁴,N⁴-dimethyl-N²-{-3-[(3-thienylmethyl)amino]-trans-cyclohexyl}quinazoline-2,4-diamine

A solution of N²-(3-amino-trans-cyclohexyl)-N⁴,N⁴-dimethyl-quinazoline-2,4-diamine (0.200 g, 0.70 mmol) and thiophene-3-carbaldehyde (0.078 g, 0.70 mmol) in MeOH:DCM (1:2, containing 6% HOAc, 8 mL) was stirred at ambient temperature for 75 min, after which a solution of NaBH₃CN (0.176 g, 2.8 mmol) in MeOH (5 mL) was added. The reaction mixture was stirred at room temperature over night after which an additional 0.5 eq. of thiophene-3-carbaldehyde was added. The temperature was raised to 50° C. and stirred at this temperature until TLC indicated that starting material was consumed. Methanol (10 mL) was added and the reaction mixture was concentrated. The residue was first purified on SiO₂ eluted with DCM:MeOH (10:1) containing 1% Et3N and then dissolved in MeCN and further purified by prep. HPLC (Eluent A: H₂O containing 0.1% TFA; Eluent B: MeCN; gradient from 10% to 90% of eluent B) to give 0.106 g (40%) of the title compound.

¹H NMR (300.1 MHz, CDCl₃), δ 7.81-7.84 (d, 1H), 7.47-7.51 (m, 2H), 7.25-7.28 (m, 1H), 7.05-7.13 (m, s, 3H), 4.43 (br, 1H), 3.85 (s, 2H), 3.29 (s, 6H), 2.93-2.97 (m, 1H), 1.27-1.99 (m, 9H). LC-MS [M+H]⁺382.

### Example 3

### N²-{-3-[(1-benzothien-3-ylmethyl)amino]-trans-cyclohexyl}-N⁴,N⁴-dimethylquinazoline-2,4-diamine

A solution of N²-(3-amino-trans-cyclohexyl)-N⁴,N⁴-dimethyl-quinazoline-2,4-diamine (0.237 g, 0.83 mmol, from Example 2b) and benzo[b]thiophene-3-carbaldehyde (0.135 g, 0.83 mmol) in MeOH:DCM (1:2, containing 1% HOAc, 20 mL) was stirred at ambient temperature for 1.5 h, after which a solution of NaBH₃CN (0.104 g, 1.66 mmol) in MeOH (4 mL) was added. The reaction mixture was stirred at room temperature until TLC indicated that starting material was consumed. Methanol (20 mL) was added and the reaction mixture was concentrated. The residue was purified on SiO₂ eluted with DCM:MeOH (98:2) containing 2% Et₃N and finally DCM:MeOH (9:1) containing 2% Et₃N to give 0.310 g (86%) of the title compound. This material was dissolved in MeCN and further purified by HPLC (Eluent A: H₂O containing 0.1% TFA; Eluent B: MeCN; gradient from 10% to 80% of eluent B) to give 0.200 g (56%) of the title compound.

¹H NMR (300.1 MHz, MeOD-d₄), δ 7.82 (d, 1H), 7.76 (m, 1H), 7.45 (t, 1H), 7.05-7.33 (m, 5H), 7.01 (t, 1H), 4.32 (br s, 1H), 3.96 (s, 2H), 3.18 (s, 6H), 2.90 (m, 1H), 1.3-2.1 (m, 8H). ¹³C NMR (75.5 MHz, MeOD-d₄), δ 164.7, 158.0, 153,3, 140.7, 138.5, 134.6, 132.3, 128.7, 128.0, 126.6, 124.2, 123.9, 123.3, 122.5, 121.4, 120.0, 111.8, 51.9, 46.0, 43.8, 40.9, 36.6, 31.3, 31.2, 19.8. LC-MS [M+H]⁺432.2.

### Example 4

### N⁴,N⁴-dimethyl-N²-(-3-{[(1-methyl-1H-indol-3-yl)methyl]amino}-trans-cyclohexyl)quinazoline-2,4-diamine

A solution of N²-(3-amino-trans-cyclohexyl)-N⁴,N⁴-dimethyl-quinazoline-2,4-diamine (0.24 g, 0.84 mmol, from Example 2b), 1-methylindole-3-carbaldehyde (0.134 g, 0.84 mmol) and NaBH(OAc)₃ (0.267 g, 1.26 mmol) in 1,2-dichloroethane (3 mL) and THF (1 mL) was stirred under a nitrogen atmosphere at ambient temperature for one day. A saturated solution of NaHCO₃ (5 mL, aq.) was added and the mixture was extracted with DCM (2×10 mL). The combined organic phases were concentrated and the residue was purified by prep. HPLC (Eluent A: H₂O containing 0.1% TFA; Eluent B: MeCN; gradient from 20% to 80% of eluent B) to give 32 mg (9%) of the title compound.

1H NMR (300.1 MHz, MeOD-d₄), δ 8.09 (d, 1H), 7.60-7.71 (m, 2H), 7.48 (d, 1H), 7.25-7.31 (m, 3H), 7.10 (t, 1H), 6.99 (t, 1H), 4.36-4.46 (m, 3H), 3.70 (s, 3H), 3.43 (s, 6H), 2.43 (br d 1H), 1.64-2.30 (m, 11H), 1.50-1.63 (m, 1H). ¹³C NMR (75.5 MHz, MeOD-d₄), δ 164.4, 138.4, 135.1, 131.8, 128.6, 128.5, 123.5, 123.3, 121.0, 120.4, 119.2, 111.9, 110.7, 105.7, 53.4, 48.0, 47.6, 42.6, 40.5, 34.2, 33.0, 30.3, 30.2, 20.6. LC-MS [M+H]⁺429.

### Example 5

### N⁴,N⁴-dimethyl-N²-((1S,3S)-3-{[2-(trifluoromethoxy)benzyl]amino}cyclohexyl)quinazoline-2,4-diamine

The title compound was prepared according to Example 6 from N²-(3-amino-trans-cyclohexyl)-N⁴,N⁴-dimethyl-quinazoline-2,4-diamine (40 mg, 0.140 mmol, from Example 2b) and 2-trifluoromethoxybenzaldehyde (27 mg, 0.142 mmol), and sodium borohydride (26 mg, 0.69 mmol). Yield: 41 mg (64%) of the title compound.

¹H NMR (500 MHz, CDCl₃), δ 7.81 (m, 1H), 7.49 (m, 1H), 7.43 (d, 1H), 7.38-7.34 (m, 2H), 7.15-7.11 (m, 2H), 7.03 (m, 1H), 4.95 (bs, 1H), 4.43 (m, 1H), 3.83 (d, 1H), 3.81 (d, 1H), 3.26 (s, 6H), 2.93 (m, 1H), 190-1.70 (m, 5H), 1.65-1.55 (m, 2H), 1.41 (m, 1H). ¹³C NMR (100 MHz, CDCl₃), δ 164.9, 157.9, 154.1, 147.9, 139.6, 132.0, 129.2, 129.0, 125.8, 125.2, 122.1, 120.7, 119.6, 118.7, 112.0, 52.1, 50.5, 45.9, 41.7, 37.7, 32.0, 31.9, 20.1. LC-MS [M+H]⁺460.1.

### Example 6

### N⁴,N⁴-dimethyl-N²-[(1S,3S)-3-({[6-(trifluoromethyl)pyridin-3-yl]methyl}amino)cyclohexyl]quinazoline-2,4-diamine

A solution of N²-(3-amino-trans-cyclohexyl)-N⁴,N⁴-dimethyl-quinazoline-2,4-diamine (35 mg, 0.123 mmol, from Example 2b), 6-(trifluoromethyl)nicotinaldehyde (22 mg, 0.125 mmol) in 2 mL of methanol was allowed to react overnight. Sodium borohydride (23 mg, 0.61 mmol) was added and the mixture was stirred for 30 min before 1 mL of 2M HCl was added. After 5 min the mixture was made alkaline by addition of 2M NaOH and 20 mL of water. The mixture was extracted three times with EtOAc and the combined organic layer was washed with water, dried over Na₂SO₄ and evaporated. The crude material was chromatographed on a prepacked 5 g Isolute Silica gel column with DCM:MeOH:TEA 100:5:1. Yield: 41 mg (75%) of the title compound.

¹H NMR (500 MHz, CDCl₃), δ 8.67 (s, 1H), 7.87 (d, 1H), 7.80 (d, 1H), 7.58 (d, 1H), 7.47 (m, 1H), 7.42 (d, 1H), 7.02 (m, 1H), 5.07 (bs, 1H), 4.39 (m, 1H), 3.92 (d, 1H), 3.89 (d, 1H), 3.25 (s, 6H), 2.93 (m, 1H), 1.90-1.70 (m, 5H), 1.65-1.53 (m, 2H), 1.40 (m, 1H). ¹³C NMR (100 MHz, CDCl₃), δ 165.2, 158.0, 153.9, 150.0, 147.4, 147.1, 146.7, 146.4, 140.1, 137.1, 132.4, 126.1, 126.0, 125.3, 123.2, 120.1, 120.3, 120.1, 117.8, 112.3, 52.6, 48.3, 46.1, 41.9, 37.7, 32.0, 20.2. LC-MS [M+H]⁺445.1.

### Example 7

### N²-{(1S,3S)-3-[(3,4-dichlorobenzyl)amino]cyclohexyl}-N⁴,N⁴-dimethylquinazoline-2,4-diamine

The title compound was prepared according to Example 6 from N²-(3-amino-trans-cyclohexyl)-N⁴,N⁴-dimethyl-quinazoline-2,4-diamine (35 mg, 0.123 mmol, from Example 2b), 3,4-dichlorobenzaldehyde (22 mg, 0.125 mmol) and sodium borohydride (23 mg, 0.61 mmol). Yield: 36 mg (66%) of the title compound.

¹H NMR (500 MHz, CDCl₃), δ 7.82 (m, 1H), 7.51 (m, 1H), 7.48-7.44 (m, 2H), 7.34 (d, 1H), 7.17 (dd, 1H), 7.05 (m, 1H), 4.43 (m, 1H), 3.80 (d, 1H), 3.78 (d, 1H), 3.28 (s, 6H), 2.94 (m, 1H), 1.90-1.70 (m, 5H), 1.65-1.55 (m, 2H), 1.41 (m, 1H). ¹³C NMR (100 MHz, CDCl₃), δ 165.2, 158.0, 147.0, 141.7, 132.4, 130.7, 130.4, 130.1, 127.6, 126.1, 125.2, 120.1, 52.4, 50.3, 46.4, 46.1, 41.2, 37.8, 32.0, 20.2, 11.7. LC-MS [M+H]⁺444.1/446.1/448.1.

Pharmacological Properties

MCH1 Receptor Radioligand Binding.

Assays were performed on membranes prepared from CHO-K1 cells expressing the human Melanin concentrating hormone receptor 1 (MCH1r). Assays were performed in a 96-well plate format in a final reaction volume of 200 μl per well. Each well contained 6 μg of membrane proteins diluted in binding buffer (50 mM Tris, 3 mM MgCl₂, 0.05% bovine serum albumin (BSA) and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at 30° C. for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a1450 Microbeta TRILUX (Wallac, Finland).

Non-specific binding was subtracted from all values determined. Maximum binding was that determined in the absence of any competitor following subtraction of the value determined for non-specific binding. Binding of compounds at various concentrations was plotted according to the equation
y=A+((B−A)/1+((C/x)ˆD)))
and IC₅₀ estimated where
A is the bottom plateau of the curve i.e. the final minimum y value
B is the top of the plateau of the curve i.e. the final maximum y value
C is the x value at the middle of the curve. This represents the log EC50 value when A+B=100
D is the slope factor.
x is the original known x values.
y is the original known y values.

The compounds exemplified herein had an IC₅₀ of less than 2 μM in the abovementioned human MCHr binding assay. Preferred compounds had an activity of less than 1 μmolar. For example, the following IC₅₀ was obtained for the compound of Example 4: 0.015 μM. Assays were also performed on membranes prepared from HEK293 cells stably expressing the rat Melanin concentrating hormone receptor 1 (MCH1r) (Lembo et al. Nature Cell Biol 1 267-271). Assays were performed in a 96-well plate format in a final reaction volume of 200 μl per well. Each well contained 5 μg of membrane proteins diluted in binding buffer (50 mM Tris, 3 mM MgCl₂, 0.05% bovine serum albumin (BSA) and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at room temperature for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a 1450 Microbeta TRILUX (Wallac, Finland).

1. A compound of formula I 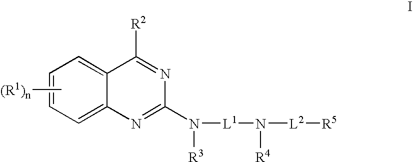
wherein
R¹ represents a) a C₁₋₄ alkoxy group optionally substituted by one or more fluoro, b) a C₁₋₄ alkyl group optionally substituted by one or more fluoro, c) halo, d) cyano, e) a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O atom, f) a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring, or g) a group —OSO₂C₁₋₄alkyl optionally substituted by one or more fluoro;
n represents 0, 1, 2 or 3;
R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H or a C₁₋₄ alkyl group;
L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O; with the proviso that L¹ does not represent 1,3-cyclopentyl or 1,4-cyclohexyl;
R⁴ represents H or a C₁₋₄ alkyl group optionally substituted by one or more of the following: fluoro or C₁₋₄ alkoxy optionally substituted by one or more fluoro;
L² represents an alkylene chain (CH₂)ₛ in which s represents 1, 2 or 3 wherein the alkylene chain is optionally substituted by one or more of the following: fluoro or C₁₋₄ alkyl;
L² may also represent a 5-6 membered carbocyclic 5-6 membered ring fused to R⁵;
R⁵ represents phenyl or naphthyl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridinyl, 5H-pyrrolo[2,3-b]pyrazinyl, 1H-pyrrolo[3,2-c]pyridinyl, 1H-pyrrolo[2,3-c]pyridinyl, 1H-pyrrolo[2,3-b]pyridinyl, 1H-indazolyl, wherein each R⁵ is optionally substituted by one or more of the following: a) cyano, b) halo, c) a C₁₋₄ alkyl group optionally substituted by one or more fluoro, d) a C₁₋₄ alkoxy group optionally substituted by one or more fluoro, e) a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, f) or by a group (CH₂)_{z}R^{z} in which z and w is 0 or 1 and R^{z} represents phenyl or a heterocyclic group selected from thienyl, pyridyl, thiazolyl, pyrazolyl, wherein each R^{z} is optionally substituted by one or more of the following: cyano, halo, a C₁₋₄ alkyl group optionally substituted by one or more fluoro, or a C₁₋₄alkoxy group optionally substituted by one or more fluoro;
as well as optical isomers and racemates thereof as well as pharmaceutically acceptable salts, thereof.

2. A compound as claimed in claim 1 in which
R¹ represents cyano or a C₁₋₄ alkoxy group optionally substituted by one or more fluoro, a C₁₋₄ alkyl group optionally substituted by one or more fluoro, halo, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
n represents 0, 1, 2 or 3;
R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H or a C₁₋₄ alkyl group;
L¹ represents a (CH₂)ₚC₅₋₆ cycloalkyl group in which p is 0 or 1 and provided that there are 3 carbon atoms between the two nitrogens bearing R³ and R⁴, respectively, wherein one of the carbons of the cycloalkyl group may be replaced by O;
R⁴ represents H or a C₁₋₄ alkyl group optionally substituted by one or more of the following: fluoro or C₁₋₄ alkoxy optionally substituted by fluoro;
L² represents an alkylene chain (CH₂)ₛ in which s represents 1, 2 or 3 wherein the alkylene chain is optionally substituted by one or more of the following: fluoro or C₁₋₄ alkyl;
L² may also represent a 5-6 membered carbocyclic 5-6 membered ring fused to R⁵;
R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, fluoro;
as well as optical isomers and racemates thereof as well as pharmaceutically acceptable salts, thereof.

3. A compound of formula IA 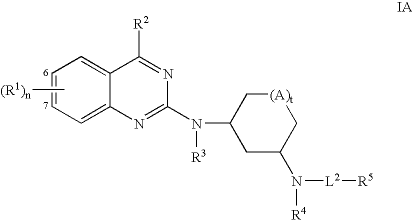
in which
R¹ represents chloro, fluoro, methoxy or a group NR^{a}R^{b} in which R^{a} and R^{b} independently represent a C₁₋₄alkyl group or R^{a} and R^{b} together with the nitrogen atom to which they are attached represent a saturated 3 to 7 membered heterocyclic ring optionally including an O;
n represents 0 or 1, and when n=1 the substituent is attached to either position 6 or 7;
R² represents H or cyano or a C₁₋₄alkyl group, a C₁₋₄alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
m represents 0 or 1;
R³ represents H;
A represents CH₂ and t is 1;
R⁴ represents H;
L² represents CH₂, C(CH₃)₂ or CF₂; and
R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro;
as well as optical isomers and racemates thereof as well as pharmaceutically acceptable salts thereof.

4. A compound of formula IB 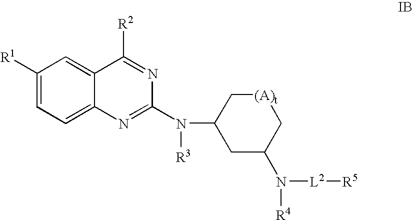
in which
R¹ represents H, cyano, methoxy, isopropoxy, dimethylamino, chloro or fluoro;
R² represents H, cyano, a C₁₋₄alkyl group optionally substituted by one or more fluoro or a C₁₋₄ alkoxy group optionally substituted by one or more fluoro, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O;
R³ represents H;
A represents CH₂ and t is 1;
R⁴ represents H;
L² represents CH₂, C(CH₃)₂ or CF₂; and
R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or 1 and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro;
as well as optical isomers and racemates thereof as well as pharmaceutically acceptable salts thereof.

5. A compound as represented by formula IC 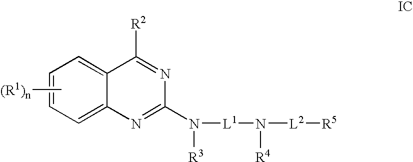
in which
R¹ represents cyano or a C₁₋₄ alkoxy group optionally substituted by one or more fluoro, a C₁₋₄ alkyl group optionally substituted by one or more fluoro, halo, a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
n represents 0, 1, 2 or 3;
R² represents H, cyano, a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H or a C₁₋₄ alkyl group;
L¹ represents a (CH₂)ₚC₇₋₁₀ cycloalkyl group in which p is 0 or 1 and in which the cycloalkyl group is fused bicyclic or bridged bicyclic provided that the two nitrogens bearing R³ and R⁴, respectively, are not linked to the same carbon atom, and wherein one of the carbons may be replaced by O;
R⁴ represents H or a C₁₋₄ alkyl group optionally substituted by one or more of the following: fluoro or C₁₋₄ alkoxy, optionally substituted by one or more fluoro;
L² represents an alkylene chain (CH₂)ₛ in which s represents 1, 2 or 3 wherein the alkylene chain is optionally substituted by one or more of the following: fluoro or C₁₋₄ alkyl;
or L² may also represent a 5-6 membered carbocyclic ring fused to R⁵;
R⁵ represents aryl or a heterocyclic group selected from thienyl, furyl, pyridyl, pyrrolyl, quinolinyl, indolyl, benzofuranyl, benzo[b]thienyl, imidazolyl, benzimidazolyl, thiazolyl, thiadiazolyl, pyrimidinyl, pyrazolyl, oxazolyl, imidazo[1,2-a]pyridine, 5H-pyrrolo[2,3-b]pyrazine, 1H-pyrrolo[3,2-c]pyridine, 1H-pyrrolo[2,3-c]pyridine, 1H-pyrrolo[2,3-b]pyridine, 1H-indazole each of which is optionally substituted by one or more of the following: cyano, halo, a C₁₋₄ alkyl group optionally substituted by one or more fluoro, a C₁₋₄ alkoxy group optionally substituted by one or more fluoro, or by a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, or a group (CH₂)_{z}R^{z} in which z is 0 or and R^{z} represents phenyl or a heterocyclic group selected from thienyl, pyridyl, thiazolyl, pyrazolyl, wherein each R^{z} is optionally substituted by one or more cyano, halo, a C₁₋₄ alkyl group optionally substituted by one or more fluoro, a C₁₋₄alkoxy group optionally substituted by one or more fluoro;
as well as optical isomers and racemates thereof as well as pharmaceutically acceptable salts thereof.

6. A compound as claimed in any one of claims 1 to 4 in which p is 0 and L¹ is 1,3-cyclohexyl.

7. A compound as claimed in any one of claims 1 to 5 in which the two nitrogen atoms are in a trans orientation on the cycloalkyl ring.

8. A compound as claimed in claim 7 wherein the stereochemistry of the cycloalkyl carbon atoms to which the nitrogen atoms are attached is S,S.

9. One or more of the following compounds:
N-(4-methylquinazolin-2-yl)-N′-(3-thienylmethyl)-trans-cyclohexane-1,3-diamine;
N⁴,N⁴-dimethyl-N²-{-3-[(3-thienylmethyl)amino]-trans-cyclohexyl}quinazoline-2,4-diamine;
N²-{-3-[(1-benzothien-3-ylmethyl)amino]-trans-cyclohexyl}-N⁴,N⁴-dimethylquinazoline-2,4-diamine;
N⁴,N⁴-dimethyl-N²-(-3-{[(1-methyl-1H-indol-3-yl)methyl]amino}-trans-cyclohexyl)quinazoline-2,4-diamine,
N⁴,N⁴-dimethyl-N²-((1S,3S)-3-{[2-(trifluoromethoxy)benzyl]amino}cyclohexyl)-quinazoline-2,4-diamine;
N⁴,N⁴-dimethyl-N²-[(1S,3S)-3-({[6-(trifluoromethyl)pyridin-3-yl]methyl}amino)-cyclohexyl]quinazoline-2,4-diamine; and
N²-{(1S,3S)-3-[(3,4-dichlorobenzyl)amino]cyclohexyl}-N⁴-N⁴-dimethylquinazoline-2,4-diamine;
and pharmaceutically acceptable salts thereof.

10. (canceled)

11. A pharmaceutical formulation comprising a compound of formula I, as defined in any one of claims 1 to 5 or in claim 9 and a pharmaceutically acceptable adjuvant, diluent or carrier.

12. (canceled)

13. A method of treating obesity, a psychiatric disorder, anxiety, an anxio-depressive disorder, depression, bipolar disorder, ADHD, a cognitive disorder, a memory disorder, schizophrenia, epilepsy, a neurological disorder, or a pain related disorder, comprising administering a pharmacologically effective amount of a compound as claimed in any one of claims 1 to 5 or in claim 9 to a patient in need thereof.

14. (canceled)

15. A process for the preparation of a compound of formula I 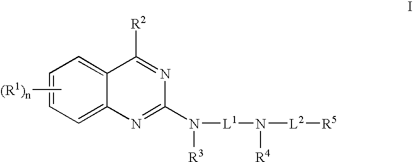
comprising reacting a compound of formula 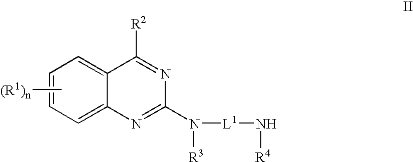
in which
R¹ represents a) a C₁₋₄ alkoxy group optionally substituted by one or more fluoro, b) a C₁₋₄ alkyl group optionally substituted by one or more fluoro, c) halo, d) cyano, e) a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O atom, f) a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring, or g) a group —OSO₂C₁₋₄alkyl optionally substituted by one or more fluoro;
R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H or a C₁₋₄alkyl group;
R⁴ represents H or a C₁₋₄alkyl group optionally substituted by one or more of the following: fluoro or C₁₋₄alkoxy optionally substituted by one or more fluoro;
L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O; with the proviso that L¹ does not represent 1,3-cyclopentyl or 1,4-cyclohexyl; and
n represents 0, 1, 2 or 3;
with a compound of formula III
R⁵-L^{2′}=O III
in which
R⁵ represents phenyl or naphthyl or a heterocyclic group selected from thienyl, furyl, pyridyl, pyrrolyl, quinolinyl, indolyl, benzofuranyl, benzo[b]thienyl, imidazolyl, benzimidazolyl thiazolyl, thiadiazolyl, pyrimidinyl, pyrazolyl, oxazolyl, imidazo[1,2-a]pyridinyl, 5H-pyrrolo[2,3-b]pyrazinyl, 1H-pyrrolo[3,2-c]pyridinyl, 1H-pyrrolo[2,3-c]pyridinyl, 1H-pyrrolo[2,3-b]pyridinyl, 1H-indazolyl, wherein each R⁵ is optionally substituted by one or more of the following: a) cyano, b) halo, c) a C₁₋₄alkyl group optionally substituted by one or more fluoro, d) a C₁₋₄ alkoxy group optionally substituted by one or more fluoro, e) a group S(O)ₐR^{y} in which a is 0, 1 or 2 and R^{y} is phenyl optionally substituted by cyano, halo, a C₁₋₄alkyl group optionally substituted by one or more fluoro or a C₁₋₄alkoxy group optionally substituted by one or more fluoro, f) or by a group (CH₂)_{z}R^{z} in which z and w is 0 or 1 and R^{z} represents phenyl or a heterocyclic group selected from thienyl, pyridyl, thiazolyl, pyrazolyl, wherein each R^{z} is optionally substituted by one or more of the following: cyano, halo, a C₁₋₄ alkyl group optionally substituted by one or more fluoro, or a C₁₋₄alkoxy group optionally substituted by one or more fluoro; and
L^{2′} represents a group which after reaction of compounds II and III gives L² on reduction, under reductive alkylation conditions.

16. A compound of formula II 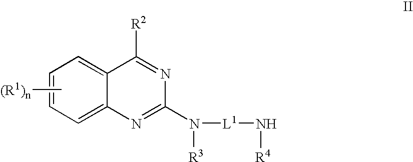
in which
R¹ represents a) a C₁₋₄ alkoxy group optionally substituted by one or more fluoro, b) a C₁₋₄ alkyl group optionally substituted by one or more fluoro, c) halo, d) cyano, e) a group NR^{a}R^{b} in which R^{a} and R^{b} independently represent H or a C₁₋₄alkyl group or R^{a} and R^{b} together with the nitrogen atom to which they are attached represent a saturated 3 to 7 membered heterocyclic ring optionally including an O atom, f) a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring, or g) a group —OSO₂C₁₋₄alkyl optionally substituted by one or more fluoro;
R² represents H or cyano or a C₁₋₄alkyl group optionally substituted by one or more fluoro or a C₁₋₄alkoxy group optionally substituted by one or more fluoro, a group NR^{a}R^{b} in which R^{a} and R^{b} independently represent H or a C₁₋₄ alkyl group or R^{a} and R^{b} together with the nitrogen atom to which they are attached represent a saturated 3 to 7 membered heterocyclic ring optionally including an O, a group CONR^{c}R^{d} in which R^{c} and R^{d} independently represent H or a C₁₋₄alkyl group or R^{c} and R^{d} together with the nitrogen atom to which they are attached represent a saturated 3 to 7 membered heterocyclic ring;
R³ represents H or a C₁₋₄alkyl group;
R⁴ represents H or a C₁₋₄alkyl group optionally substituted by one or more of the following: fluoro or C₁₋₄alkoxy optionally substituted by one or more fluoro;
L¹ represents a (CH₂)ₚC₃₋₁₀ cycloalkyl group in which p is 0 or 1 and in which the cycloalkyl group may be monocyclic or bicyclic and optionally may be bridged provided that the two nitrogens bearing R³ and R⁴, respectively, are not linked to the same carbon atom, and wherein one of the carbons may be replaced by O; with the proviso that L¹ does not represent 1,3-cyclopentyl or 1,4-cyclohexyl; and
n represents 0, 1, 2 or 3.

17. A method of treating obesity, type II diabetes, or Metabolic syndrome comprising administering a pharmacologically effective amount of a compound as claimed in any one of claims 1 to 5 or in claim 9 to a patient in need thereof.

18. A method of preventing type II diabetes comprising administering a pharmacologically effective amount of a compound as claimed in any one of claims 1 to 5 or in claim 9 to a patient in need thereof.
